(19) United States
(12) Patent Application Publication
(10) Pub. No.: US 2018/0339124 A1
(43) Pub. Date: Nov. 29, 2018

(54) HYDROGEN GAS SUPPLYING APPARATUS

(71) Applicant: NIHON KOHDEN CORPORATION, Shinjuku-ku, Tokyo (JP)

(72) Inventors: Mitsushi HYOGO, Shinjuku-ku, Tokyo (JP); Tohru KURATA, Shinjuku-ku, Tokyo (JP); Shinya NAGATA, Shinjuku-ku, Tokyo (JP)

(73) Assignee: NIHON KOHDEN CORPORATION, Shinjuku-ku, Tokyo (JP)

(21) Appl. No.: 15/778,089

(22) PCT Filed: Nov. 15, 2016
(86) PCT No.: PCT/JP2016/004889

§ 371 Date: May 22, 2018

(30) Foreign Application Priority Data
Dec. 1, 2015 (JP) 2015-234379

(51) Int. Cl. A61M 16/12; A61M 16/00

(52) U.S. Cl. CPC A61M 16/12; A61M 16/024; A61M 2202/02; A61M 2230/43; A61M 2205/581; A61M 2205/583; A61M 2230/20; A61M 2205/3334

(57) ABSTRACT

A hydrogen gas supplying apparatus includes a switching section configured to output, in accordance with switch instructions, one of a hydrogen-containing gas that contains hydrogen and a non-hydrogen-containing gas that does not contain hydrogen, an output section through which the gas that has been output from the switching section is supplied to a subject, and a controller configured to send the switch instructions to the switching section in accordance with an elapsed time and to control an amount of hydrogen that is output from the switching section. The controller controls an amount of hydrogen output per unit time during a first period from a start of switching from the non-hydrogen-containing gas to the hydrogen-containing gas to be greater than an amount of hydrogen output per unit time during a second period successively following the first period.

### TECHNICAL FIELD

The presently disclosed subject matter relates to an apparatus for supplying hydrogen gas, and in particular, to a hydrogen gas supplying apparatus configured to intermittently administer hydrogen gas.

### BACKGROUND ART

Recently, the use of hydrogen and hydrogen water in various treatments including inflammation treatment and ischemic reperfusion is attracting attention (see, e.g., WO2007/021034A1).

It has been reported that hydrogen has effects such as hydroxy radical reduction, ghrelin secretagogue, and fibroblast growth factor (FGF) 21 induction. Hydrogen is the smallest molecule, and can easily reach all the internal organs and cells forming the organs. Since hydrogen has the above-described functions, hydrogen exerts effects such as suppression of death of cells due to oxidant stress or radioactive rays, that of ischemic reperfusion injury, anti-inflammation, that of diabetes, and that of Parkinson disease. Moreover, it is said that a treatment and health management method using hydrogen have high safety to humans (see, e.g., Ikuroh Ohsawa, “Suiso Bunshi Igaku no Genjyo to Tenbo (Molecular Hydrogen Medicine: Current Status and Future Challenges)”, Feb. 8, 2011, Kiso Roka Kenkyu (Biomedical Gerontology), Vol. 35 No. 1, pp. 1-8). Under such circumstances, a hydrogen-containing gas and hydrogen-containing water attract much attention in the health promotion industry and medical sites (related articles published on May 8, 2007 in the morning papers of Kanagawa Shimbun, The Mainichi Newspapers, The Asahi Shimbun, and The Yoraiuri Shimbun).

Ito et al. disclose a method for enhancing treatment effects by intermittent administration of hydrogen, and teach that, when hydrogen was intermittently administered, high effects were obtained in four of six cases (Ito, et al., “Drinking hydrogen water and intermittent hydrogen gas exposure, but not lactulose or continuous hydrogen gas exposure, prevent 6-hydroxydopamine-induced Parkinson's disease in rats”, Medical Gas Research 2012, 2:15).

To intermittently administer hydrogen, a hydrogen-containing gas (first gas) and a non-hydrogen-containing gas (second gas, for example, the air) are alternately administered. However, the administration may be affected by a residual gas in a supply path (e.g., a tube) for supplying hydrogen, and there is a possibility that hydrogen at a desired concentration cannot be administered. For example, in a case where a hydrogen-containing gas containing 2% hydrogen and treatment air (0% hydrogen) are alternately output, when the output of the gas containing 2% hydrogen is started, the treatment air remains in the tube so that it is difficult to administer 2% hydrogen into the body. Failing to administer a desired concentration of hydrogen may result in an insufficient effect of intermittent administration of hydrogen.

### SUMMARY

Illustrative aspects of the present invention provide a hydrogen gas supplying apparatus that can effectively perform intermittent administration of hydrogen.

According to an aspect of the present invention, a hydrogen gas supplying apparatus includes a switching section configured to output, in accordance with switch instructions, one of a hydrogen-containing gas that contains hydrogen and a non-hydrogen-containing gas that does not contain hydrogen, an output section through which the gas that has been output from the switching section is supplied to a subject, and a controller configured to send the switch instructions to the switching section in accordance with an elapsed time and to control an amount of hydrogen that is output from the switching section. The controller controls an amount of hydrogen output per unit time during a first period from a start of switching from the non-hydrogen-containing gas to the hydrogen-containing gas to be greater than an amount of hydrogen output per unit time during a second period successively following the first period.

### BRIEF DESCRIPTION OF DRAWINGS

FIG. 1 is a block diagram of an example of a hydrogen gas supplying apparatus.

FIG. 2 illustrates operations of the hydrogen gas supplying apparatus of FIG. 1.

FIG. 3 is a block diagram illustrating a configuration of a hydrogen gas supplying apparatus according to an exemplary embodiment of the present invention.

FIG. 4 illustrates operations of the hydrogen gas supplying apparatus of FIG. 3.

FIG. 5 is a block diagram illustrating a configuration of a hydrogen gas supplying apparatus according to another exemplary embodiment of the present invention.

FIG. 6 illustrates operations of a hydrogen gas supplying apparatus according to another exemplary embodiment of the present invention.

FIG. 7 illustrates an operation of the hydrogen gas supplying apparatus associated with the operations illustrated in FIG. 6.

FIG. 8 is a block diagram illustrating a configuration of a hydrogen gas supplying apparatus according to another exemplary embodiment of the present invention.

FIG. 9 is a block diagram illustrating a configuration of a hydrogen gas supplying apparatus according to another exemplary embodiment of the present invention.

FIG. 10 is a block diagram illustrating a configuration of a hydrogen gas supplying apparatus according to another exemplary embodiment of the present invention.

FIG. 11 illustrates operations of a hydrogen gas supplying apparatus according to another exemplary embodiment of the present invention.

### DESCRIPTION OF EXEMPLARY EMBODIMENTS

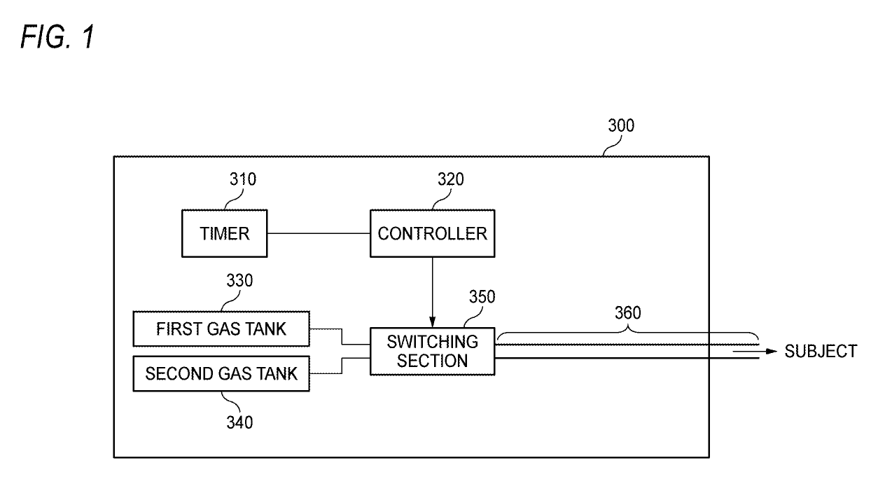

First, a problem associated with an intermittent administration of hydrogen will be again described. FIG. 1 is a block diagram of an example of a hydrogen gas supplying apparatus 300 configured to intermittently administers a hydrogen gas.

The hydrogen gas supplying apparatus 300 has a timer 310, a controller 320, a first gas tank 330, a second gas tank 340, a switching section 350, and an output section 360. When the timer 310 reaches a predetermined time, the controller 320 outputs a gas switch signal to the switching section 350. The first gas tank 330 is a tank which holds a first gas. The first gas contains x% hydrogen (4>x>0). The second gas tank 340 is a tank which holds a second gas. The second gas is a gas which does not contain hydrogen (e.g., the air). The switching section 350 supplies only one of the first gas and the second gas to the output section 360, by using the gas switch signal as a trigger. The output section 360 supplies the gas output from the switching section 350, to the subject. The output section 360 may be configured by selecting arbitrary members from a gas tube, a reservoir bag, a mask, and the like.

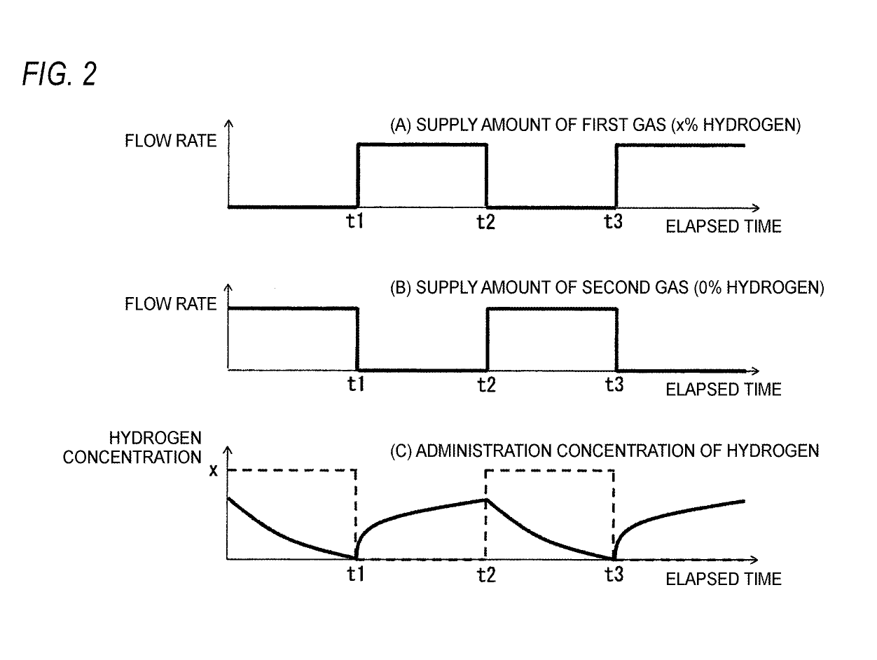

FIG. 2 includes (A) a chart showing the flow rate of the first gas (x% hydrogen), (B) a chart showing the flow rate of the second gas (0% hydrogen), and (C) a chart showing the concentration of hydrogen which is to be administered to the subject (solid line), and an ideal concentration of hydrogen (broken lines). The charts (A) to (C) share the same time axis.

Here, the capacity of the output section 360 is indicated by V (L), and the flow rate of the gas which is to be administered to the subject is indicated by Q (L/sec). In this case, the hydrogen concentration indicated by the solid line in the chart (C) of FIG. 2 is expressed by following Expression (1) (e in Expression (1) is the base of natural logarithm):

C(t)=A x(1−e^{t/T})

T=V/Q [sec] Expression (1)

The switching section 350 switches the outputs of the first gas and the second gas at timings t1, t2, and t3. As illustrated in the chart (C) of FIG. 2, however, the hydrogen concentration is changed in a gradual manner due to the capacity (V) of the output section 360 and the gas flow rate (Q). Moreover, the hydrogen concentration is affected by the second gas remaining in the output section 360, and fails to reach x%. As such, when the two kinds to gas (first gas and second gas) are alternately supplied as described above, they mutually affect each other so that the hydrogen concentration is changed in a gradual manner. That is, in a period during which the hydrogen is to be supplied (t1 to t2), hydrogen is supplied at a concentration that is lower than a desired concentration (x%), and in a period during which the hydrogen is not supposed to be supplied (t2 to t3), a small amount of hydrogen is supplied. As a result, sufficient effect of intermittent administration cannot be achieved. Hereinafter, configurations for solving this problem will be described.

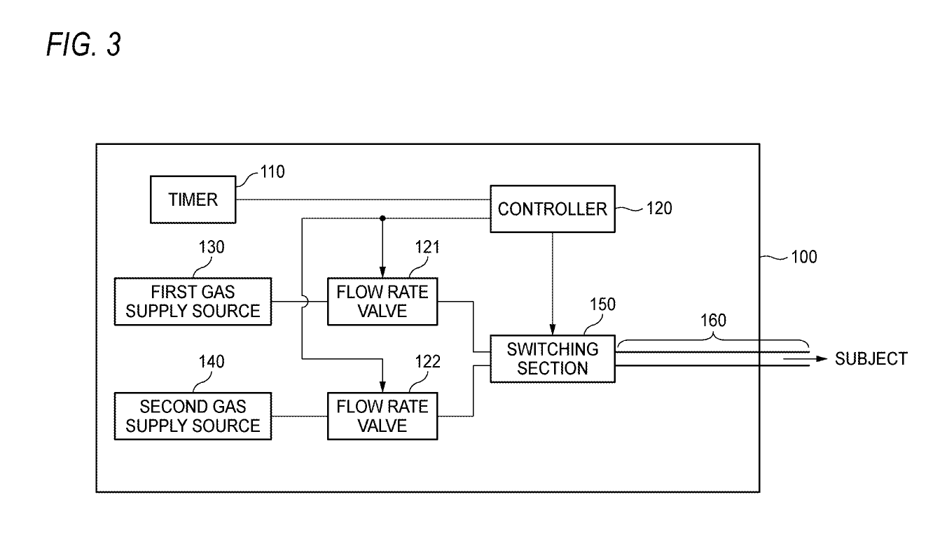

Exemplary embodiments of the present invention will now be described with reference to the drawings. FIG. 3 is a block diagram illustrating a configuration of a hydrogen gas supplying apparatus 100 according to an exemplary embodiment of the present invention. The hydrogen gas supplying apparatus 100 may be a stand-alone apparatus which supplies a hydrogen gas to the subject, or a built-in apparatus which is built in an artificial respirator or the like.

The hydrogen gas supplying apparatus 100 may include a timer 110, a controller 120, a flow rate valve 121, a flow rate valve 122, a first gas supply source 130, a second gas supply source 140, a switching section 150, and an output section 160. The hydrogen gas supplying apparatus 100 intermittently supplies hydrogen to the subject who is connected to the output section 160.

The timer 110 is configured to measure time by using, for example, a quartz oscillator and the like.

The first gas supply source 130 supplies a first gas which is a hydrogen-containing gas that contains hydrogen. The first gas is a treatment gas. The first gas supply source 130 may be a gas cylinder which is filled with the first gas, or configured so as to mix internally generated hydrogen with another gas to produce the first gas. In the case of the configuration where hydrogen is internally generated, the first gas supply source 130 may have a configuration which is provided with, for example, an electrolysis unit that electrolyzes pure water to generate hydrogen. In the following description, it is assumed that the hydrogen containing concentration of the first gas is x% (0<x<4).

The second gas supply source 140 supplies a second gas which is a non-hydrogen-containing gas that does not contain hydrogen. The term “does not contain hydrogen” means that the gas does not contain hydrogen in an amount at which hydrogen exhibits treatment effects, and has a concept including a case where the gas contains about 0.01% of hydrogen. For example, the second gas may be treatment air or the like. The second gas supply source 140 may be, for example, a gas cylinder which is filled with the second gas.

The switching section 150 supplies one of the first gas and the second gas to the output section 160 in accordance with switch instructions (e.g., switch signals) from the controller 120. The switching section 150 may be, for example, a solenoid valve or the like.

The output section 160 administers the gas output from the switching section 150, to the subject. The output section 160 may be, for example, a gas tube or the like. The output section 160 may be connected to a reservoir bag, a mask for a respirator, or the like.

The controller 120 monitors the timer 110 to acquire an elapsed time. The controller 120 sends the switch instructions to the switching section 150 at given timings to switch the gas output. When it is set that the gas output is to be switched every 30 seconds, for example, the controller 120 monitors the elapsed time, and outputs the switch instruction every 30 second to the switching section 150. The timings at which the gas output is to be switched may be changed through an operating section (e.g., an interface such as buttons arranged on a housing of the hydrogen gas supplying apparatus 100).

The controller 120 controls the flow rate valves 121, 122 to control the output amounts of the first gas and the second gas. The flow rate valve 121 is adjusting means for adjusting the output amount of the first gas which is output from the first gas supply source 130. The flow rate valve 122 is adjusting means for adjusting the output amount of the second gas which is output from the second gas supply source 140. Alternatively, the flow rate adjustment may be realized by a configuration which does not use a valve.

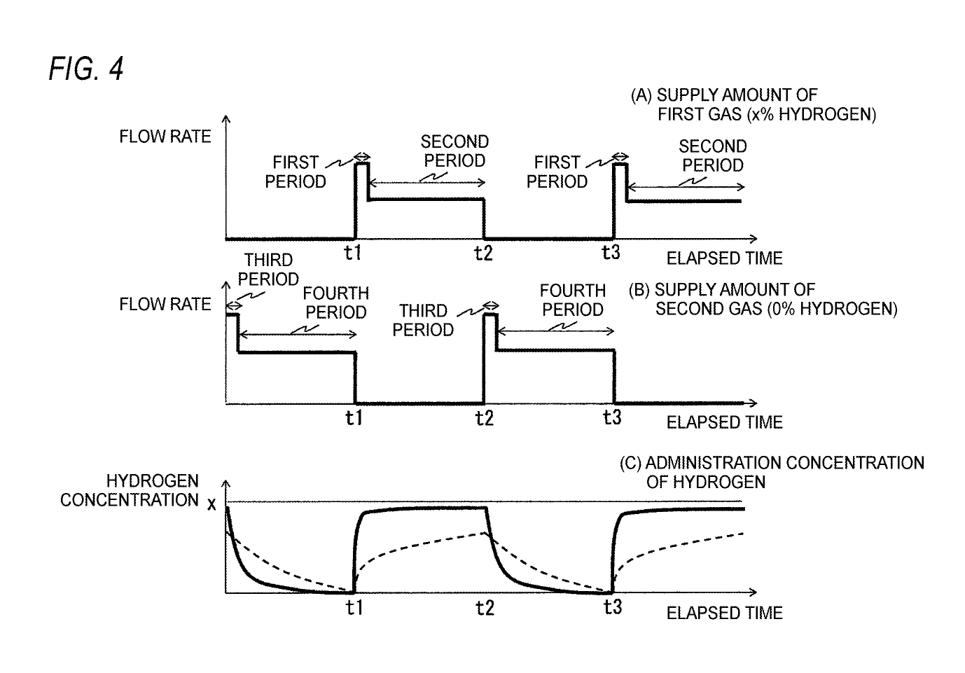

When switching from the first gas to the second gas, the controller 120 controls the flow rate of the first gas per unit time during a given time period (first period) from the start of the switching to be greater than the flow rate of the first gas per unit time during a successively following time period (second period). When switching from the second gas to the first gas, the controller 120 controls the flow rate of the second gas per unit time during a given time period (third period) from the start of the switching to be greater than the flow rate of the second gas per unit time during a successively following time period (fourth period). The details of these controls will be described with reference to FIG. 4.

FIG. 4 includes (A) a chart showing the flow rate of the first gas (x% hydrogen), (B) a chart showing the flow rate of the second gas, and (C) a chart showing the concentration of hydrogen which is to be administered to the subject (the solid line) together with the concentration of hydrogen (the broken lines) shown in the chart (C) of FIG. 2. In the following description, it is assumed that it is requested to intermittently administer x% hydrogen to the subject. The charts (A) to (C) of FIG. 4 share the same time axis.

The controller 120 controls the outputs of the first gas and the second gas so as to be switched at predetermined timings (t1, t2, and t3 in the present example). This causes only one of the first gas and the second gas to be output from the switching section 150.

The controller 120 provides a state in which the output amount of hydrogen per unit time is large during the given time period (first period) from the timing at which the output is switched to the first gas. That is, the controller 120 controls the amount of hydrogen per unit time during the first period immediately after the switching to be larger than the amount of hydrogen per unit time during the successively following second period. The first period is the time period from the timing at which the output is switched to the first gas (hydrogen-containing gas). Preferably, the first period starts immediately after the timing at which the output is switched to the first gas. However, the first period may allow a slight time lag after the timing at which the switching starts (e.g., the first period may start in about 0.5 to 1 second after the switching).

The second period is a period after the end of the first period, and during which the first gas is supplied. The controller 120 controls the flow rate of the first gas to adjust the output amount of hydrogen. In the case where the first period is 1 second, for example, the flow rate of the first gas in the first period is larger than that of the first gas for 1 second (the unit time period=1 second) in the second period. Therefore, the output amount of hydrogen per unit time during the first period is greater than the output amount of hydrogen per unit time during the second period. This control may be implemented by adjusting the flow rate valve 121.

The controller 120 provides a state in which the flow rate of the second gas (non-hydrogen-containing gas) output per unit time is large during the given time period (third period) from the timing at which the output is switched to the second gas. That is, the controller 120 controls the flow rate of the second gas per unit time during the third period after the switching to be greater than the flow rate of the second gas per unit time in the successively following fourth period. The third period is the time period from the timing at which the output is switched to the second gas. Preferably, the third period starts immediately after the timing at which the output is switched to the second gas. However, the third period may allow a slight time lag after the timing at which the switching starts (e.g., the third period may start in about 0.5 to 1 second after the switching). The fourth period is a period after the end of the third period, and during which the second gas is supplied. In the case where the third period is 1 second, for example, the flow rate of the second gas in the third period is larger than that of the second gas for 1 second (the unit time period=1 second) in the fourth period. This control may be implemented by adjusting the flow rate valve 122.

At timing t1, the flow rate of the first gas is rapidly increased (see the chart (A) of FIG. 4). This causes the hydrogen concentration (the solid line) of the gas which is to be administered to the subject is sharply raised at timing t1 as compared with the reference example (the broken line) (see the chart (C) of FIG. 4).

At tuning t2, furthermore, the flow rate of the second gas is rapidly increased (see the chart (B) of FIG. 4). This causes the hydrogen concentration (the solid line) is sharply lowered at timing t2 as compared with the reference example (the broken line) (see the chart (C) of FIG. 4).

As such, when switching the gas, the flow rate is controlled to be increased for the given time period from the timing of the switching, whereby it is possible to cancel the effect of the residual gas.

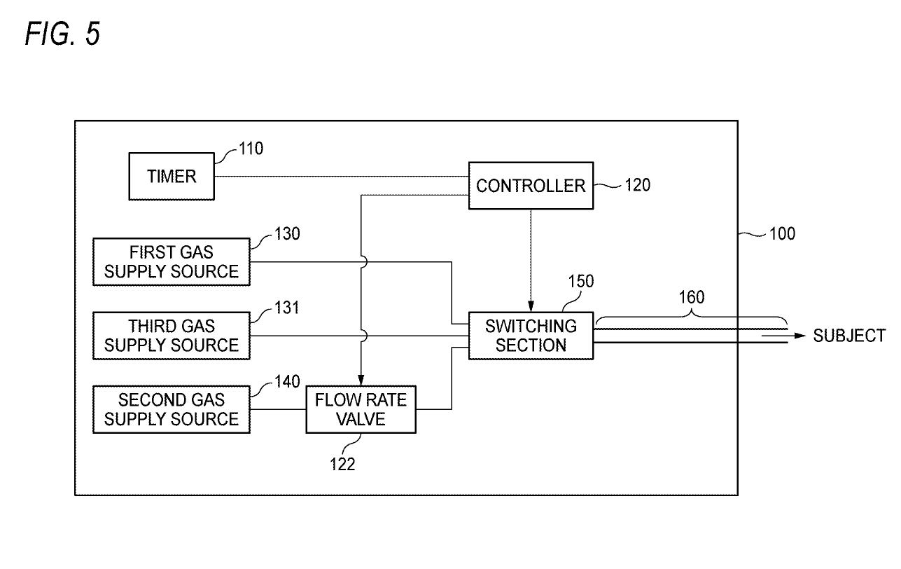

In the configuration of FIG. 3, the effect of the residual gas is cancelled by controlling the flow rate of the first gas. However, the invention is not limited to this. FIG. 5 illustrates a modified example. The configuration of FIG. 5 includes a third gas supply source 131. The flow rate control is not performed on the first and third gas supply sources 130, 131, and the gas is supplied from the respective sources at a constant flow rate.

The third gas supply source 131 outputs a third gas containing hydrogen. The third gas contains hydrogen at a higher concentration than the first gas. The third gas supply source 131 may be a gas tank filled with the third gas, or may be configured to internally produce hydrogen (e.g., an electrolysis unit).

The controller 120 controls the switching section so as to output the third gas in the first period (the given time period after the start of switching from the non-hydrogen-containing gas to the hydrogen-containing gas). Then, the controller 120 controls the switching section so as to output the first gas in the second period succeeding the first period. With respect to the output of the second gas, the controller 120 may perform a control which is substantially identical with the control illustrating in the chart (B) of FIG. 4.

That is, the controller 120 may control the amount of hydrogen output per unit time during the first period to be greater than the amount of hydrogen output per unit time during the second period. In the configuration of FIG. 3, the controller 120 implements this control by adjusting the flow rate of the same hydrogen-containing gas (first gas). In the configuration of FIG. 5, the controller 120 implements this control by switching the output of two kinds of hydrogen-containing gas (first gas and third gas) between the first period and the second period.

According to the hydrogen gas supplying apparatus 100 described above, the controller 120 provides a state in which the output amount of hydrogen per unit time during the given time period (first period) after the switching of the output from the second gas (non-hydrogen-containing gas) to the first gas (hydrogen-containing gas) is greater than the output amount of hydrogen per unit time during the successively following time period (second period) (see the chart (A) of FIG. 4). Since the amount of hydrogen that is output during the first period is large, the effect of the second gas remaining in the output section 160 can be rapidly eliminated. Therefore, the problem in that the amount of hydrogen administered to the subject is reduced at the gas switching can be relieved. Namely, hydrogen can be effectively administered to the subject from the start (timings t1, t3 in the chart (C) of FIG. 4) of the output of the first gas.

The controller 120 controls the flow rate of the second gas per unit time during the given time period (third period) from the start of the switching from the first gas to the second gas to be greater than the flow rate of the second gas per unit time during the successively following time period (fourth period) (see the chart (B) of FIG. 4). Therefore, the effect of hydrogen remaining in the output section 160 can be immediately cancelled (in other words, hydrogen is immediately administered to the subject, and hydrogen can be eliminated from the output section 160). In the hydrogen gas supplying apparatus 100, consequently, it is possible to immediately provide a period when hydrogen is not administered. That is, the desired state of intermittent administration can be immediately attained, and treatment effects can be enhanced.

Next, a configuration of a hydrogen gas supplying apparatus 100 of according to another exemplary embodiment of the present invention will be described. This hydrogen gas supplying apparatus 100 is configured such that, when the output cycles of the hydrogen-containing gas and the non-hydrogen-containing gas are to be alternately repeated, the output amount of hydrogen is gradually increased. Hereinafter, points which are different from the exemplary embodiment of FIGS. 3 and 4 will be described. In the following description, the processing sections indicated by the same names and reference signs as those of the exemplary embodiment of FIGS. 3 and 4 operate in the same or similar manner unless otherwise described (the same shall apply to the subsequently described exemplary embodiments).

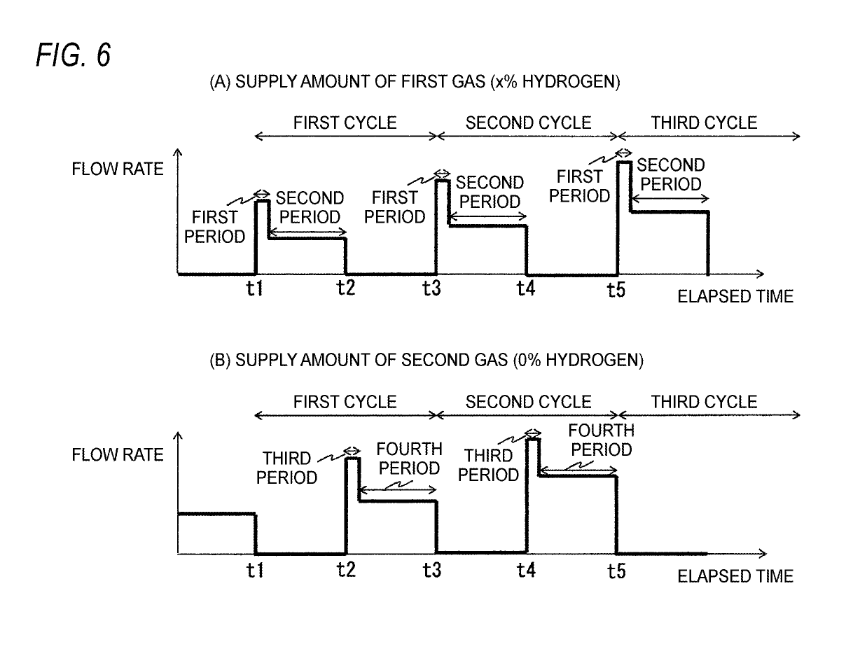

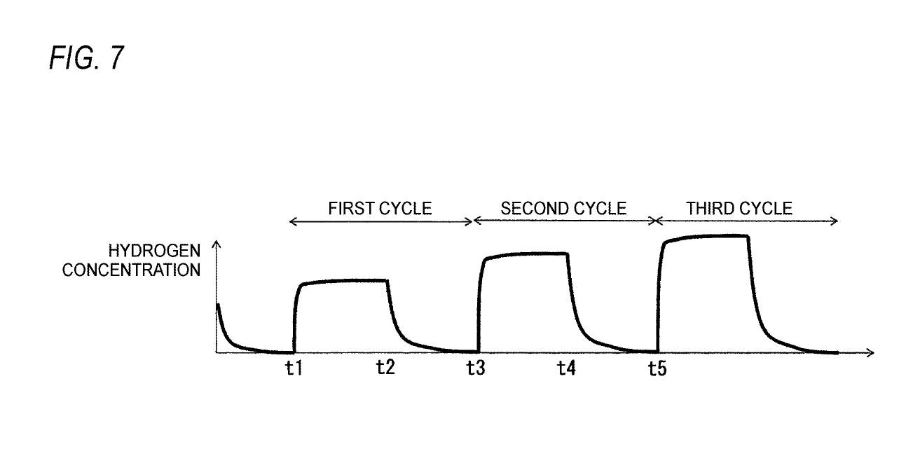

The configuration of the hydrogen gas supplying apparatus 100 is identical with that of the exemplary embodiment of FIG. 3, and therefore its detailed description is omitted. Hereinafter, an example of a control performed by the controller 120 will be described with reference to FIGS. 6 and 7.

FIG. 6 includes (A) a chart showing the flow rate of the first gas (x% hydrogen), and (B) a chart showing the flow rate of the second gas (0% hydrogen). FIG. 7 is a chart showing the concentration of hydrogen which is to be administered to the subject. In the charts (A) and (B) of FIG. 6 and the chart of FIG. 7 share the same time axis.

Here, the sum of the time period from the start of the output of the hydrogen-containing gas (first gas) to the end (from the start of the first period to the end of the second period), and the time period from the start of the output of the non-hydrogen-containing gas (second gas) to the end (from the start of the third period to the end of the fourth period) is referred to as “one cycle.” In the example of FIGS. 6 and 7, three cycles (first to third cycles) are illustrated.

The controller 120 controls the amount of hydrogen which is output in a certain cycle, so as to be larger than that of hydrogen which is output in the immediately previous cycle. Referring to the chart (A) of FIG. 6, the controller 120 controls the flow rate of the first gas in the second cycle so as to be larger than that of the first gas in the first cycle. Moreover, the controller 120 controls the flow rate of the first gas in the third cycle so as to be larger than that of the first gas in the second cycle. That is, the controller 120 controls the output amount of hydrogen so as to be gradually increased each time the cycle is repeated. Similarly, the controller 120 controls the flow rate of the second gas so as to be gradually increased each time the cycle is repeated (see the chart (B) of FIG. 6). The flow rate control may be realized by a flow rate control in which the flow rate valves 121, 122 are used.

The concentration of hydrogen which is supplied to the subject by the flow rate control will be described with reference to FIG. 7. As described above, the output amount of hydrogen is increased each time the cycle is repeated. This causes also the hydrogen concentration of the gas which is supplied to the subject, to be raised each time the cycle is repeated (FIG. 7).

In the case where hydrogen is intermittently administered to the subject, the body of the subject may be accustomed by the hydrogen treatment. According to the hydrogen gas supplying apparatus 100 described above, however, the amount of hydrogen administered to the subject is gradually increased (FIG. 7). Therefore, treatment effects due to hydrogen can be sufficiently obtained even in the case where the body of the subject is gradually accustomed by the hydrogen treatment.

According to another exemplary embodiment of the present invention, a hydrogen gas supplying apparatus 100 is configured to notify the hydrogen administration status. The features of this hydrogen gas supplying apparatus 100 that are different from those of the foregoing exemplary embodiments will be described below.

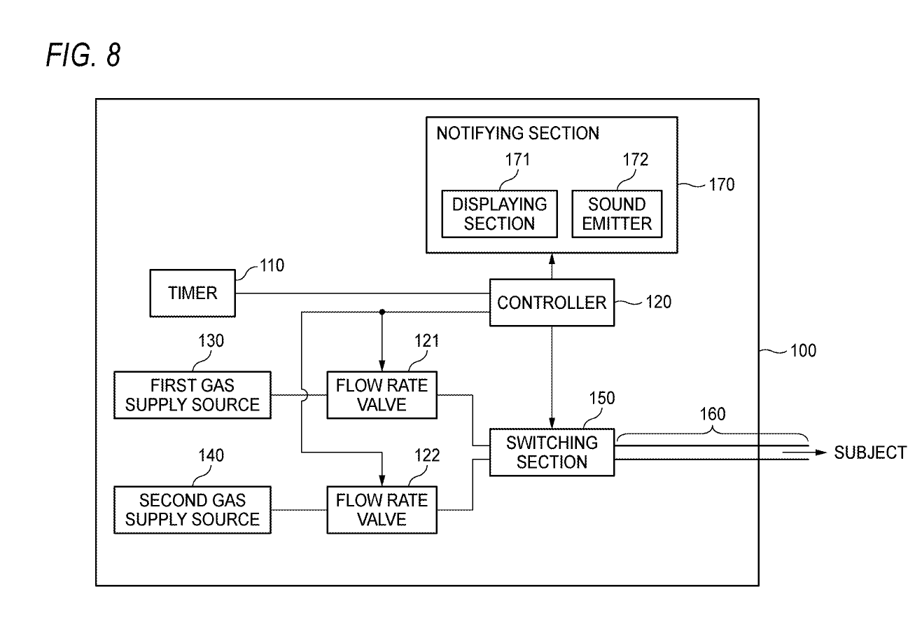

FIG. 8 is a block diagram illustrating a configuration of the hydrogen gas supplying apparatus 100. The hydrogen gas supplying apparatus 100 has a notifying section 170 in addition to the configuration of FIG. 3. The notifying section 170 visually or audibly notifies of the supplying state of hydrogen and an abnormal state. The notifying section 170 may include a displaying section 171 and a sound emitter 172.

The displaying section 171 visually displays various kinds of information. The displaying section 171 may include, for example, a display device and/or an indicator lamp arranged on the housing of the hydrogen gas supplying apparatus 100. The sound emitter 172 outputs various notification sounds such as an alarm sound.

The controller 120 informs the notifying section 170 of the flow rate (corresponding to the chart (A) of FIG. 4) of the first gas, the flow rate (corresponding to the chart (B) of FIG. 4) of the second gas, the hydrogen concentration (corresponding to the chart (C) of FIG. 4) calculated from Expression (1) above by using the capacity of the output section 160 and the like, the timing at which the output of hydrogen is started, the timing at which the output of hydrogen is ended, and the like. That is, the controller 120 notifies the notifying section 170 of the output status of hydrogen.

The notifying section 170 performs various notifications based on the output status of hydrogen provided by the controller 120. For example, the displaying section 171 displays an indication prompting inhalation immediately before the start of the output of hydrogen, or during the output. The displaying section 171 may display a graph of the hydrogen concentration like the chart (C) of FIG. 4.

Similarly, the sound emitter 172 may output a beep sound or voice message prompting preparation for inhalation at the timing when the output of hydrogen is started (e.g., the timings t1, t3 in the chart (C) of FIG. 4). The sound emitter 172 may further output a beep sound or voice message at the timing when the output of hydrogen is started (e.g., the timing t2 in the chart (C) of FIG. 4).

According to the configuration, the subject can know the timing when hydrogen is output, and the output status. Since the subject can know the output timing and the like, it is possible to avoid a situation such as that where the subject forgets inhalation of hydrogen. Therefore, the hydrogen gas supplying apparatus 100 can surely administer hydrogen to the subject.

According to another exemplary embodiment of the present invention, a hydrogen gas supplying apparatus 100 may be configured to detect a gas in the output section 160 by a sensor, and to control the gas output based on a result of the detection. Hereinafter, the features that are different from those of the foregoing exemplary embodiments will be described.

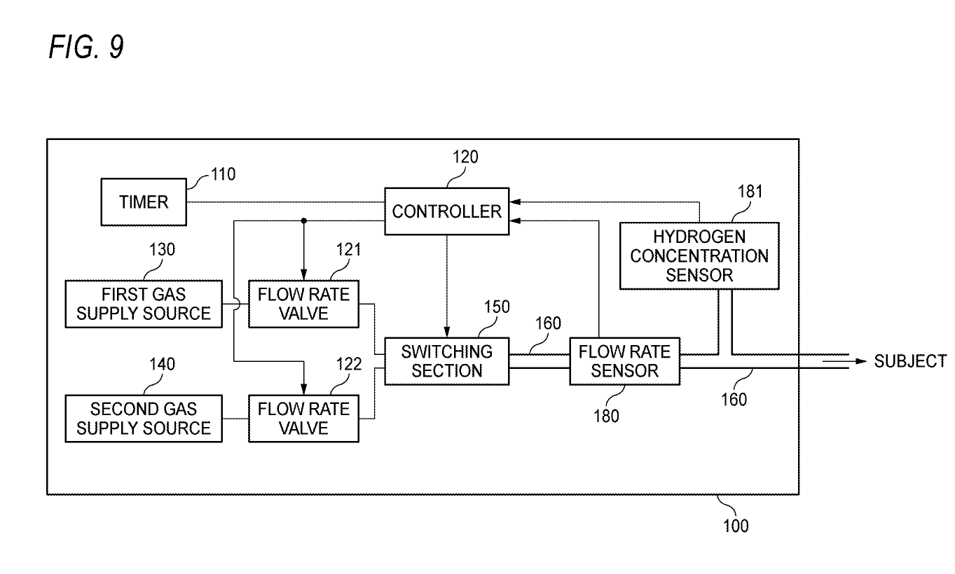

FIG. 9 is a block diagram illustrating a configuration of the hydrogen gas supplying apparatus 100. The hydrogen gas supplying apparatus 100 includes a flow rate sensor 180 and a hydrogen concentration sensor 181 in addition to the configuration of the exemplary embodiment of FIG. 3.

The flow rate sensor 180 detects the flow rate of a gas flowing through the output section 160. The flow rate sensor 180 may be a sensor for measuring the flow rate of a gas, such as a differential pressure flow rate sensor, an ultrasonic flow rate sensor, or a thermal flow rate sensor. The flow rate sensor 180 sends the detected flow rate of the gas to the controller 120.

The hydrogen concentration sensor 181 detects the hydrogen concentration of the gas flowing through the output section 160. The hydrogen concentration sensor 181 may have a general configuration used for measuring the concentration of hydrogen. For example, the sensor may be a thermal conductivity concentration measuring apparatus. The hydrogen concentration sensor 181 sends the detected hydrogen concentration of the gas to the controller 120.

The controller 120 controls the gas outputs (in detail, adjusts the flow rate valves 121, 122) based on the gas flow rate detected by the flow rate sensor 180, and the hydrogen concentration detected by the hydrogen concentration sensor 181. When the detected hydrogen concentration is lower than a desired (preset) hydrogen concentration, for example, the controller 120 adjusts the flow rate valve 121 to increase the flow rate of the first gas. When the detected hydrogen concentration is higher than the desired (preset) hydrogen concentration, the controller 120 adjusts the flow rate valve 122 to increase the flow rate of the second gas. Similarly, when the detected gas flow rate is smaller than a desired (preset) gas flow rate, the controller 120 adjusts at least one of the flow rate valves 121, 122 to increase the gas flow rate (the flow rate of at least one of the first gas and the second gas).

Alternatively, the hydrogen gas supplying apparatus 100 may be configured so as to be provided with only one of the flow rate sensor 180 and the hydrogen concentration sensor 181.

As described above, in accordance with the detection result of the flow rate sensor 180, the controller 120 adjusts the flow rate of at least one of the first gas and the second gas. When the flow rate is smaller or larger as compared with a scheduled gas administration amount, therefore, the gas administration amount can be quickly adjusted.

As described above, in accordance with the detection result of the hydrogen concentration sensor 181, the controller 120 adjusts the flow rate of at least one of the first gas and the second gas to change the hydrogen concentration. When the hydrogen concentration is deviated from a scheduled hydrogen concentration, therefore, the hydrogen concentration can be quickly adjusted. Also in the configuration of FIG. 5, the controller 120 may adjust the hydrogen concentration by, for example, adjusting the length of the first period.

According to another exemplary embodiment of the present invention, a hydrogen gas supplying apparatus 100 is configured to detect hydrogen concentration on a body surface of a subject or hydrogen concentration in blood or expiration of the subject, and to control (adjust) a gas output based on a result of the detection. Hereinafter, the features that are different from those of the foregoing exemplary embodiments will be described.

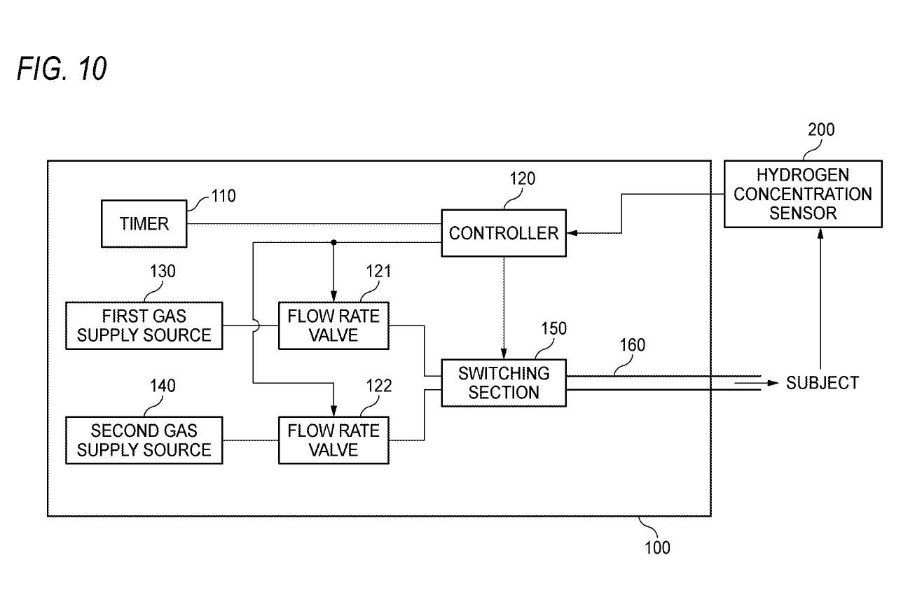

FIG. 10 is a block diagram illustrating a configuration of the hydrogen gas supplying apparatus 100. The hydrogen gas supplying apparatus 100 is configured to control (adjust) the gas output in accordance with the result of the detection conducted by a hydrogen concentration sensor 200 attached to the subject.

The hydrogen concentration sensor 200 is for estimating the amount (concentration) of hydrogen ingested into the body of the subject. The hydrogen concentration sensor 200 detects the hydrogen concentration in at least one of the expiration, blood, and the body surface. The hydrogen concentration sensor 200 may be configured to be adhered to the body surface, to obtain blood and to detect the concentration of hydrogen in the blood, and/or to obtain expiration of the subject and to detect the concentration of hydrogen in the expiration.

The hydrogen concentration which is detected by the hydrogen concentration sensor 200 is used as an index for the amount of hydrogen ingested into the body of the subject. For example, in a case where 2% hydrogen is administered and the hydrogen concentration in the expiration is 0.2%, it can be estimated that hydrogen of the amount corresponding to 1.8% is ingested into the body. The hydrogen concentration sensor 200 sends the detected hydrogen concentration to the controller 120. The hydrogen concentration sensor 200 may send the hydrogen concentration to the controller 120 via a communicating section (not shown) inside the hydrogen gas supplying apparatus 100, by means of a wireless communication or the like.

The controller 120 adjusts the flow rates of the first gas and the second gas based on the hydrogen concentration received from the hydrogen concentration sensor 200. For example, in a case where 2% hydrogen is administered and the hydrogen concentration in the expiration is 0.1%, most of the administered hydrogen is ingested into the body, and therefore it can be expected that the treatment effects may be further enhanced by administering a little more amount of hydrogen. Therefore, the controller 120 may control the flow rate of the first gas so as to be increased to raise the hydrogen concentration.

According to the exemplary embodiment described above, the amount of hydrogen which is to be output is adjusted based on the hydrogen concentration of at least one of the expiration, blood, and body surface of the subject. While checking the treatment effects on the subject, therefore, the hydrogen gas supplying apparatus 100 can supply an adequate amount of hydrogen to the subject.

According to another exemplary embodiment of the present invention a hydrogen gas supplying apparatus 100, administration periods when hydrogen is intermittently administered, and intermission periods when the gas supply is not performed are provided. Hereinafter, the features that are different from those of the foregoing exemplary embodiments will be described.

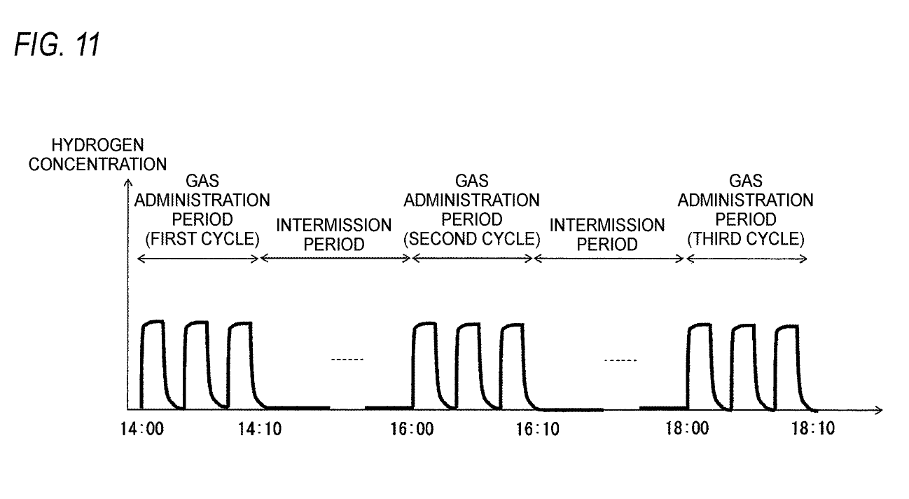

The configuration of the hydrogen gas supplying apparatus 100 is identical with that of exemplary embodiment of FIG. 3, and therefore its detailed description will be omitted. Hereinafter, another example of a control performed by the controller 120 will be described with reference to FIG. 11.

The user (mainly the doctor or the like) inputs timings when hydrogen is to be intermittently administered, through an operating section (e.g., buttons or touch panel that is disposed on the hydrogen gas supplying apparatus 100) which is not shown. For example, the user inputs intermittent administrations of hydrogen in ten-minute periods which start from 12:00, 14:00, and 16:00, respectively. The setting contents are stored in a storage section which is not shown.

FIG. 11 illustrates an example of transition of the concentration of hydrogen which is administered to the subject in the case where the above-described setting is performed. The controller 120 reads out the setting contents of intermittent administrations from the storage section, and controls the outputs of the first gas and the second gas in accordance with the setting. As illustrated in FIG. 11, the intetmittent administration of hydrogen is executed in each of ten-minute periods which start from 14:00, 16:00, and 18:00, respectively. Namely, the controller 120 alternately repeats three gas administration periods (first to third cycles) and two intermission periods (periods when the gas supply is not performed).

Usually, the administration amount of hydrogen per day is limited. Similarly with drug administration, when hydrogen is administered a plurality of times at intervals, therefore, treatment effects are enhanced. According to the hydrogen gas supplying apparatus 100 described above, since the intermission periods are provided as described above, a plurality of hydrogen administrations can be realized, and high treatment effects can be realized.

While the present invention has been described with reference to certain exemplary embodiments thereof, the scope of the present invention is not limited to the exemplary embodiments described above, and it will be understood by those skilled in the art that various changes and modifications may be made therein without departing from the scope of the present invention as defined by the appended claims.

This application is based on Japanese Patent Application No. 2015-234379 filed on Dec. 1, 2015, the entire content of which is incorporated herein by reference.

1. A hydrogen gas supplying apparatus comprising:
a switching section configured to output, in accordance with switch instructions, one of a hydrogen-containing gas that contains hydrogen and a non-hydrogen-containing gas that does not contain hydrogen;
an output section through which the gas that has been output from the switching section is supplied to a subject; and
a controller configured to send the switch instructions to the switching section in accordance with an elapsed time and to control an amount of hydrogen that is output from the switching section,
wherein the controller controls an amount of hydrogen output per unit time during a first period from a start of switching from the non-hydrogen-containing gas to the hydrogen-containing gas to be greater than an amount of hydrogen output per unit time during a second period successively following the first period.

2. The hydrogen gas supplying apparatus according to claim 1, wherein the controller controls a flow rate of the non-hydrogen-containing gas output per unit time during a third period from a start of switching from the hydrogen-containing gas to the non-hydrogen-containing gas to be greater than a flow rate of the non-hydrogen-containing gas output per unit time during a fourth period successively following the third period.

3. The hydrogen gas supplying apparatus according to claim 1, further comprising:
a first gas supply source configured to supply a first gas, the first gas being the hydrogen-containing gas; and
a second gas supply source configured to supply a second gas, the second gas being the non-hydrogen-containing gas,
wherein the controller controls a flow rate of the first gas per unit time during the first time to be greater than a flow rate of the first gas per unit time during the second period.

4. The hydrogen gas supplying apparatus according to claim 1, wherein further comprising:
a first gas supply source configured to supply a first gas, the first gas being the hydrogen-containing gas;
a second gas supply source configured to supply a second gas, the second gas being the non-hydrogen-containing gas; and
a third gas supply source configured to supply a third gas, the third gas being higher in hydrogen concentration than the first gas,
wherein the controller causes the third gas to be output from the switching section during the first period, and causes the first gas to be output from the switching section during the second period.

5. The hydrogen gas supplying apparatus according to claim 1, wherein the controller causes an amount of hydrogen that is output in a certain cycle to be increased from an amount of hydrogen that is output in an immediately previous cycle, each cycle being a sum of a time period from a start to an end of the output of the hydrogen-containing gas and a time period from a start to an end of the output of the non-hydrogen-containing gas.

6. The hydrogen gas supplying apparatus according to claim 1, wherein further comprising a notifying section configured to perform a visible or audible notification,
wherein the controller sends an output status of hydrogen to the notifying section, and
the notifying section performs the notification based on the output status of hydrogen received from the controller.

7. The hydrogen gas supplying apparatus according to claim 1, wherein further comprising a flow rate sensor configured to detect a gas flow rate in the output section,
wherein the controller controls the output of at least one of the hydrogen-containing gas and the non-hydrogen-containing gas in accordance with the gas flow rate detected by the flow rate sensor.

8. The hydrogen gas supplying apparatus according to claim 1, wherein further comprising a hydrogen concentration sensor configured to detect a hydrogen concentration in the output section,
wherein the controller controls the output of at least one of the hydrogen-containing gas and the non-hydrogen-containing gas in accordance with the hydrogen concentration detected by the hydrogen concentration sensor.

9. The hydrogen gas supplying apparatus according to claim 1, wherein the controller controls the output of the hydrogen-containing gas in accordance with a result of a detection of at least one of a hydrogen concentration on a body surface of the subject, a hydrogen concentration in blood of the subject, and a hydrogen concentration in an expiration of the subject.

10. The hydrogen gas supplying apparatus according to claim 1, wherein the controller provides an administration period during which hydrogen is intermittently administered, and an intermission period during which the gas administration is not performed.
